(19) United States
(12) Patent Application Publication
(10) Pub. No.: US 2023/0190409 A1
(43) Pub. Date: Jun. 22, 2023

(54) System to Generate Staged Orthodontic Aligner Treatment

(71) Applicant: 3M INNOVATIVE PROPERTIES COMPANY, St. Paul, MN (US)

(72) Inventors: Alexandra R. Cunliffe, St. Paul, MN (US); Guruprasad Somasundaram, St. Paul, MN (US); Elisa J. Collins, Oakdale, MN (US); Nitsan Ben-Gal Nguyen, Apple Valley, MN (US); Benjamin D. Zimmer, Hudson, WI (US)

(21) Appl. No.: 17/928,761

(22) PCT Filed: May 10, 2021
(86) PCT No.: PCT/IB2021/053962

§ 371 Date: Nov. 30, 2022

Related U.S. Application Data
(60) Provisional application No. 63/033,887, filed on Jun. 3, 2020.

(51) Int. Cl. A61C 7/00

(52) U.S. Cl. CPC A61C 7/002; A61C 2007/004

(57) ABSTRACT

Methods for generating stages for a portion of orthodontic aligner treatment for a digital 3D model of teeth in malocclusion. The methods generate a subset of stages of setups among a complete set of stages of setups for aligner treatment of the teeth. The subset of stages can be selected from a complete set of stages, based upon a target intermediate setup, or sequentially generated from one stage to the next in the subset. Aligners for the subset of stages of setups can then be manufactured without having to make a complete set of aligners. A method to generate a setup for the aligner treatment compares the digital 3D model of teeth in malocclusion to a plurality of setups for historical cases of teeth in malocclusion that have undergone aligner treatment.

### BACKGROUND

The goal of the orthodontic treatment planning process is to determine where the post-treatment positions of a person's teeth (setup state) should be, given the pre-treatment positions of the teeth in a malocclusion state. This process is typically performed manually using interactive software and is a very time-consuming process. Furthermore, the course of treatment can change, requiring changes to the setup state. A need thus exists for an algorithm to generate a subset of the setup stages between the initial and final setups

### SUMMARY

A computer-implemented method for generating stages for a portion of orthodontic aligner treatment includes receiving a digital 3D model of teeth in malocclusion and generating a subset of stages of setups among a complete set of stages of setups for aligner treatment of the teeth.

A computer-implemented method for generating a setup for orthodontic aligner treatment includes receiving a digital 3D model of teeth in malocclusion. The method uses a machine learning model that has been trained using historic setups to generate a proposed final or intermediate setup for the digital 3D model of teeth in malocclusion.

### BRIEF DESCRIPTION OF THE DRAWINGS

FIG. 1 is a diagram of a system for receiving and processing digital models based upon 3D scans.

FIG. 2 is a flow chart of a method to generate staged aligner treatment.

FIG. 3 is a flow chart of a model development and training method for generating a final setup for aligner treatment.

FIG. 4 is a flow chart of a model deployment method for generating a final setup for aligner treatment.

FIG. 5 illustrates a digital final setup in a side view.

### DETAILED DESCRIPTION

### Overview

Embodiments include a computerized system to generate stages for a portion of a complete aligner treatment. The system takes as input a digital three-dimensional (3D) model of a malocclusion. Optional input includes treatment guidelines such as a number of stages, amount of tooth movement, or treatment strategy, or any combination thereof. The digital 3D model then undergoes any necessary preprocessing, which may include data cleanup, tooth segmentation, and tooth coordinate system identification. Next, the first N stages of treatment are generated from the preprocessed scan. The user of the system can optionally make modifications to the treatment before sending the digital data to a manufacturing process for tray manufacturing.

Embodiments also include a deep learning model to automatically generate a digital setup from the malocclusion positions of teeth. This process can be divided into two steps: model development and training, and model deployment. During model training, many digital 3D models of patients' malocclusions and setups are input into a deep learning model, which is optimized to learn patterns that minimize the difference between predicted and actual setups. During model deployment, the trained deep learning model is used to generate a setup prediction for new case data.

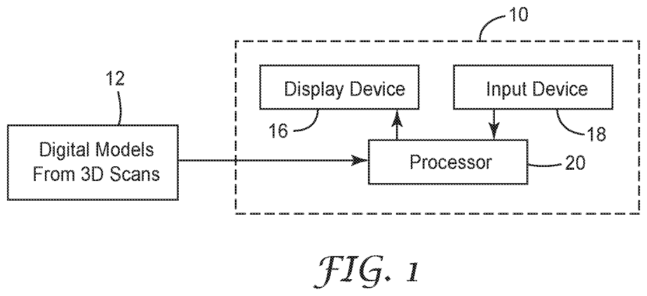

FIG. 1 is a diagram of a system 10 for receiving and processing digital 3D models based upon intra-oral 3D scans. System 10 includes a processor 20 receiving digital 3D models of teeth (12) from intra-oral 3D scans or scans of impressions of teeth. System 10 can also include an electronic display device 16, such as a liquid crystal display (LCD) device, and an input device 18 for receiving user commands or other information. Systems to generate digital 3D images or models based upon image sets from multiple views are disclosed in U.S. Pat. Nos. 7,956,862 and 7,605,817, both of which are incorporated herein by reference as if fully set forth. These systems can use an intra-oral scanner to obtain digital images from multiple views of teeth or other intra-oral structures, and those digital images are processed to generate a digital 3D model representing the scanned teeth. System 10 can be implemented with, for example, a desktop, notebook, or tablet computer. System 10 can receive the 3D scans locally or remotely via a network.

### Stages for Aligner Treatment

A typical aligner treatment planning workflow is based on designing an ideal final position of teeth (final setup), then designing a set of stages used to manufacture trays that will move the teeth from the initial setup to final setup. In some alternative workflows, it may be preferable to design only a subset of aligner trays that achieve a certain treatment goal. For example, an orthodontist may want to use a different workflow to design a few stages of treatment to create space between teeth prior to attempting more complex movements.

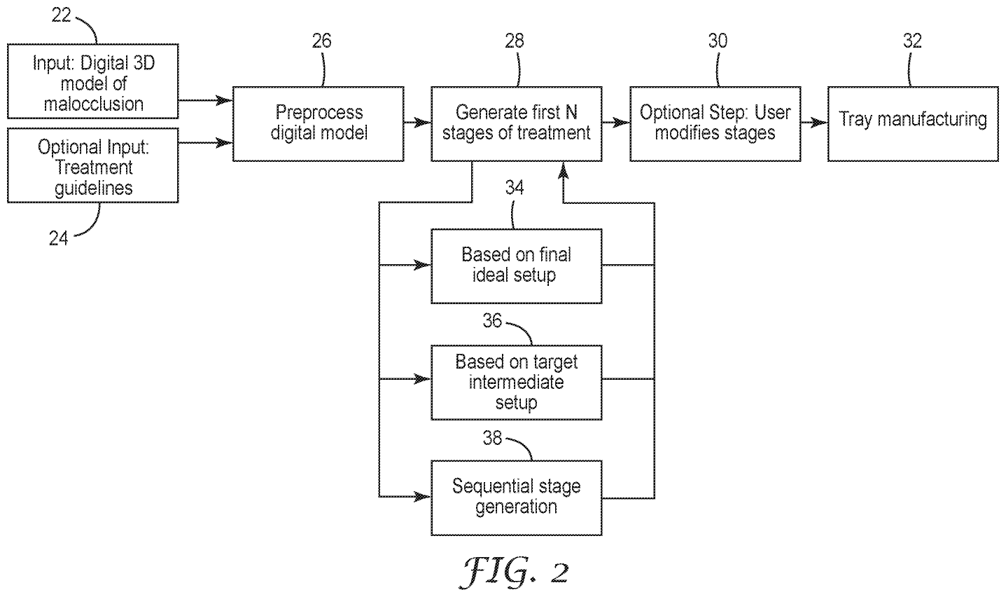

FIG. 2 is a flow chart of a method to generate staged aligner treatment. This method can be implemented in software or firmware for execution by processor 20. The method receives as input a digital 3D model of teeth in malocclusion (22) and optionally input treatment guidelines such as those identified above (step 24). The digital 3D model of malocclusion is preprocessed (step 26) and the first N stages of treatment are generated (step 28). Several algorithmic methods for creating only a portion of a complete aligner treatment, N stages, include approaches based on an ideal final setup (step 34) or a target intermediate setup (step 36), or sequential stage generation (step 38). After generating the N stages of treatment, a user can optionally modify the stages (step 30). The aligner trays are manufactured based upon the generated N stages (step 32).

The following are algorithmic methods to generate and possibly manufacture only a portion of a complete aligner treatment based upon these approaches.

Approach 1: Based on a final ideal setup (step 34).

One approach to this problem involves creating the final ideal setup and all intermediate stages. Only a subset of stages (either the first N of M stages, or stages up to a treatment goal among the final setup and intermediate stages) would then be selected and manufactured into the corresponding aligners.

Approach 2: Based on a target intermediate setup (step 36).

Rather than using a final ideal setup, a target intermediate setup that achieves a set of desired tooth movements can be created. All intermediate stages between the malocclusion and target setup could then be generated and manufactured. This target intermediate setup could either be created manually by a user (doctor or technician), or created algorithmically. Exemplary algorithmic approaches include the following:

Algorithm 1: Given a set of movements that would be desirable to achieve, an algorithm could apply these movements to any malocclusion. For example, if expansion is desired to create space, the algorithm would apply an input amount of crown torque and/or bodily expansion to the teeth. This algorithm can use a rule-based approach to generate the setups from malocclusion to the target intermediate setup. An example of a rule-based approach to generate setups is disclosed in PCT Patent Application Publication No. WO 2019/069191, which is incorporated herein by reference as if fully set forth.

Algorithm 2: One optimization algorithm that creates setups based on optimizing a set of metrics subject to some constraints in described in PCT Patent Application Publication No. WO 2020/026117, which is incorporated herein by reference as if fully set forth. These metrics and constraints can be modified to create target intermediate setups. For example, the algorithm can increase the constraint on tooth movement, which would result in a setup that moves teeth less than the amount allowed for the final setup. The algorithm can also modify the metrics to penalize certain types of movement that may be difficult to achieve at first (e.g., root torque) and promote desirable movement types (e.g., expansion). The optimization algorithm can be run with these modified constraints and metrics to create an optimal target setup. Table 1 provides exemplary pseudocode for generating final setups for this optimization-based approach.

TABLE 1
Algorithm for optimization-based final setup generation

```
Given a state (arrangement of digital teeth in the mouth) and a required
maximum score
(MaxScore):
Score = ScoringFunction(state) # compute score of current state
If (Score > MaxScore): #stopping criteria, exit routine once Score is less
than or equal to MaxScore
  NewState = Perturb(State) # apply a Perturb function to move one or
more teeth in the state
  NewState = Constrain(NewState) # apply a Constrain function to adjust
  tooth positions so that they meet constraints
  NewState Score = ScoringFunction(NewState)
  If (NewStateScore < Score): # check if score has improved
    State = NewState
Constrain function = operation performed on state to ensure state meets
requirements (e.g. tooth movement limits)
Scoring function:
$Score ( X ) = {\sum }_{i = 1}^{n \_ metrics} {w}_{i} {P}_{i} ( {x}_{i} )$
X represents the vector of metrics computed for a state. Pᵢ is a penalty
function that computes the error or penalty given a value of a metric and
acceptable levels for that metric. For instance, this penalty could be the
absolute difference between the median of acceptable values of this metric
and the current instance value, or the squared difference, and so on. wᵢ is
the weight associated with the penalty for that metric, which is a scalar
quantity. The weights w allow the scoring function to weight different
metrics more or less. For example, one scoring function might focus
on overall correctness, while another would focus on tooth alignment.
```

The method for this approach can modify metrics (change the penalty term in the Scoring function) and/or constraints (change the Constrain function) to create target intermediate setups. For example:

1. Constraints: Increase the constraint on tooth movement, which would result in a setup that moves teeth less than the amount allowed for the final setup. The Constrain function would move the teeth in the current state to a position in which the movement between the maloccluded state and the current state is less than a certain amount.

2. Metrics: Penalize certain types of movement that may be difficult to achieve at first (e.g., root torque). The penalty term in the scoring function would measure the amount of tooth movement for these types of movement. Promote desirable movement types (e.g., expansion). The penalty term would penalize movements that are less than a threshold amount by measuring how much less the current movement is compared to an ideal amount.

The optimization algorithm can be run with these modified constraints and metrics to create an optimal target setup.

Algorithm 3: Given a set of intermediate target setups from previously treated patients, a machine learning model can be trained to generate intermediate target setups. Given a malocclusion for a new patient case, this trained model can then be used to generate a custom intermediate target setup for the new case.

Approach 3: Sequential stage generation (step 38).

Given a set of teeth in a malocclusion position, a subsequent set of teeth that are displaced from the initial malocclusion (Stage 1) may be generated. From Stage 1, Stage 2 can be generated, and more stages generated until the desired number of stages are generated. Exemplary algorithmic approaches to generate stages sequentially are detailed below.

Algorithm 1: Given a set of movements that would be desirable to achieve and that respect per-stage tooth movement limits, an algorithm could apply these movements to the malocclusion as well as any subsequent stage that has been generated.

Algorithm 2: The constraints on tooth movement detailed in the optimization algorithm above (Approach 2, Algorithm 2, Constraints) can be modified to reflect per-stage tooth movement limits. Specifically:

Constraints: Increase the constraint on tooth movement, which would result in a new state that moves teeth no more than the amount allowed between consecutive states. The Constrain function would move the teeth in the current state to a position in which the movement between the previous state and the current state is less than a certain amount.

The optimization algorithm may then be run on the malocclusion or any stage to generate the next stage.

Algorithm 3: Given a setup of intermediate stages from previously treated patients, a machine learning model can be trained to generate the next intermediate stage from the current stage. For this Algorithm 3, a target setup need not be generated; rather, the stages are generated in sequence from one to the next.

### Machine Learning for Setup Generation

An optimization based approach for determining final setups is described in PCT Patent Application Publication No. WO 2020/026117. This approach includes a method of arriving at a final setup by trying to optimize scores of quality metrics related to a good final setup such as midline, class relationship, alignment, etc. This method can be altered more directly to suit the needs of a particular protocol change, need or preference. For example, if it is desired that the root movement should be reduced, a penalty or a weight on the cost function related to root movement can be increased. However, programming more complex movements using this algorithm can be challenging.

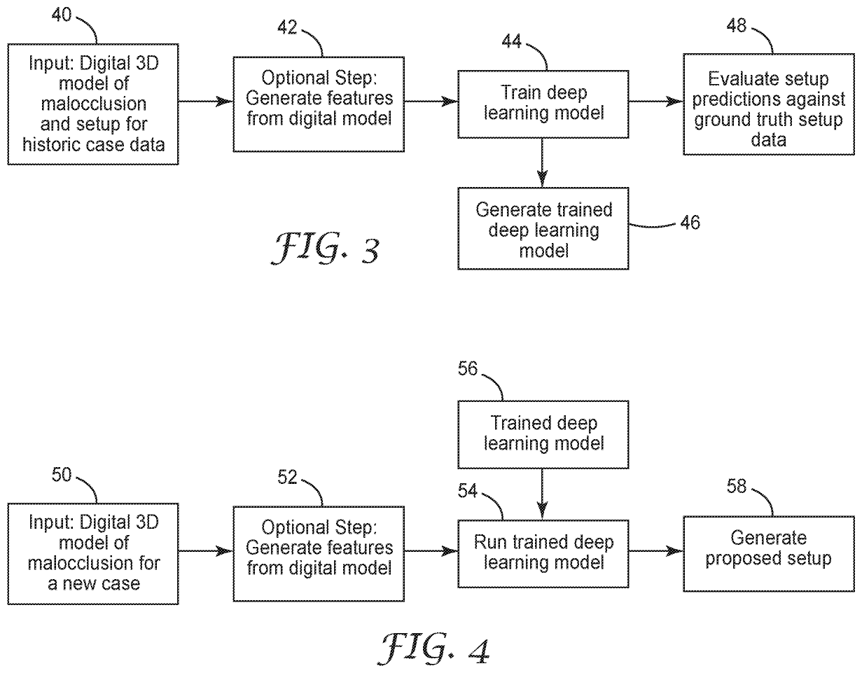

FIG. 3 is a flow chart of a model development and training method for generating a final setup for aligner treatment. FIG. 4 is a flow chart of a model deployment method for generating a final setup for aligner treatment. These methods can be implemented in software or firmware for execution by processor 20. The development and training method receives as input a digital model of malocclusion and a setup for historic case data (step 40). Features from the 3D model are optionally generated (step 42). The method trains a deep learning model (step 44) to generate a trained deep learning model (step 46) and evaluates setup predictions against ground truth setup data (step 48). The deployment method receives as input a digital 3D model of malocclusion for a new case (step 50). Features from the 3D model are optionally generated (step 52). The method runs the trained deep learning model 56 generated from the method of FIG. 3 (step 54) to generate a proposed setup (step 58).

As more data is acquired, machine learning methods and particularly deep learning methods start performing on par or exceed the performance of explicitly programmed methods. Deep learning methods have the significant advantage of removing the need for hand-crafted features as they are able to infer useful features using a combination of non-linear functions of higher dimensional latent or hidden features, directly from the data through the process of training While trying to solve the final setup problem, directly operating on the malocclusion 3D mesh can be desirable. Methods such as PointNet, PointCNN, and MeshCNN can address this problem.

Alternatively, deep learning from the methods of FIGS. 3 and 4 can be applied to processed mesh data. For example, the deep learning can be applied after the mesh of the full mouth has been segmented to individual teeth, and canonical tooth coordinate systems have been defined. At this stage, useful information such as tooth positions, orientations, dimensions of teeth, gaps between teeth, and others is available. Tooth positions are cartesian coordinates of a tooth's canonical origin location which is defined in some semantic context. Tooth orientations can be represented as rotation matrices, unit quaternions, or another 3D rotation representation such as Euler angles with respect to a global frame of reference. Dimensions are real valued 3D spatial extents and gaps can be binary presence indicators or real valued gap sizes between teeth especially in instances when certain teeth are missing. Deep learning methods can be made to use various heterogeneous feature types easily. In this feature space, even a simple multilayer perceptron model is useful and might be sufficient. Alternatively, methods not limited to but including regularized autoencoder, variational autoencoder, or generative adversarial neural networks can also be used. The goal is predicting the tooth positions and orientations of teeth in setup position using the features available in mal position. Special loss functions to weight the error in positions and orientations are desirable owing to the difference in scale and sensitivities. Additionally, scaling can be applied during the training process. These models are trained using a training set, compared on a validation set to select the best model. The best model(s) are evaluated for out-of-set or generalization performance.

Customization of these models to perform different types of treatment plans can be achieved easily by training the model with data belonging to that category, for example data of a particular doctor or data from cases where anterior teeth only were expanded.

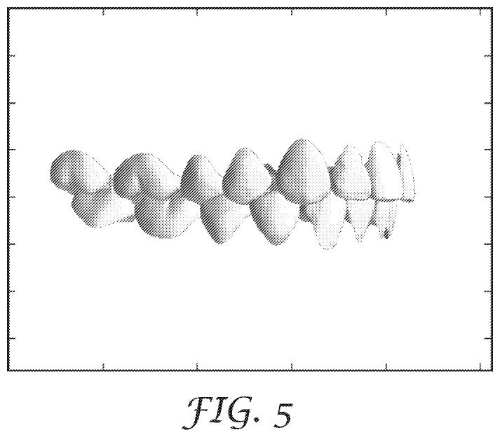

FIG. 5 illustrates a side view of the digital final setup from the deep learning approach. The digital setup shown in FIG. 5 can be displayed, for example, in a user interface on electronic display device 16.

### Fixed Teeth and Pinned Teeth

In generating the setup, it is often required that certain teeth not be moved. If a tooth is marked as fixed, it may not be moved from its original position in the patient's mouth. If it is marked as pinned, it may not be moved from a certain position. The deep learning algorithm described herein learns to generate setups that are similar to setups made by others, with no guarantee that the fixed and pinned teeth remain unmoved. One possible approach to keeping fixed and pinned teeth in place is to adjust lambdas in the machine learning loss function so that it heavily penalizes movement of teeth that a technician has specified as either being fixed or pinned. In such a loss function, teeth are divided in to two groups—those that are fixed or pinned (indicated by values of 1.0 in the input vector), and those that are not (indicated by values of 0.0 in the input vector). During training, loss is calculated separately for each group by calculating the mean-squared-error (MSE) of the difference in tooth positions between the ground-truth positions as placed by a technician, and the positions generated by the neural network during training. The MSE pertaining to the fixed and pinned teeth is then multiplied by a lambda weighting factor when calculating total loss. The equations for this approach are provided in Table 2.

```
TABLE 2
  fixed_pinned_loss=MSE(ground_truth_fixed_pinned_teeth_positions,
neural_network_fixed_pinned_teeth_positions)
  non_fixed_pinned_loss=MSE(ground_truth_non_fixed_pinned_teeth_positions,
neural_network_non_fixed_pinned_teeth_positions)
  total_loss = lambda * fixed_pinned_loss + non_fixed_pinned_loss
```

This approach does not guarantee that the fixed and pinned teeth are not moved, so after a setup is generated by the neural network, the fixed and pinned teeth are moved back to their correct positions. The desired result is that the fixed and pinned teeth have moved a small enough amount such that moving them back into position does not cause a large problem with collisions with the other teeth. Increasing the value of lambda during training should not largely affect the positions of teeth generated by the deep learning algorithm.

1. A computer-implemented method for generating stages for a portion of orthodontic aligner treatment, the method comprising:
receiving a digital 3D model of teeth in malocclusion; and
generating a subset of stages of setups among a complete set of stages of setups for aligner treatment of the teeth.

2. The method of claim 1, wherein generating the subset of stages comprises:
generating the complete set of stages of setups;
selecting the subset of stages from the complete set of stages; and
manufacturing only the subset of stages into corresponding aligners.

3. The method of claim 1, wherein generating the subset of stages comprises:
generating a set of stages of setups from the digital 3D model of teeth in malocclusion to a target intermediate setup representing desired movement of the teeth for the portion of a complete treatment; and
selecting the subset of stages from the set of stages.

4. The method of claim 3, further comprising receiving the target intermediate setup from a user.

5. The method of claim 3, further comprising generating the target intermediate setup based upon a desired set of movements of the teeth.

6. The method of claim 3, further comprising generating the target intermediate setup based upon metrics, constraints, or both metrics and constraints relating to movement of the teeth.

7. The method of claim 3, further comprising generating the target intermediate setup based upon a set of target intermediate setups from previously treated patients.

8. The method of claim 1, wherein generating the subset of stages comprises sequentially generating the subset of the stages, wherein each stage of the subset is generated based upon a most-recent previous stage.

9. The method of claim 8, further comprising generating each stage of the subset by applying a set of desired movements to the most-recent previous stage.

10. The method of claim 8, further comprising generating each stage of the subset by applying per-stage tooth movement limits to the most-recent previous stage.

11. The method of claim 8, further comprising generating each stage of the subset based upon intermediate setups from previously treated patients.

12. The method of claim 1, further comprising receiving treatment guidelines for the digital 3D model of teeth in malocclusion.

13. A computer-implemented method for generating a setup for orthodontic aligner treatment, the method comprising:
receiving a digital 3D model of teeth in malocclusion; and
using a machine learning model that has been trained using historic setups to generate a proposed final or intermediate setup for the digital 3D model of teeth in malocclusion.

14. The method of claim 13, further comprising generating features from the digital 3D model of teeth before the using step.

15. The method of claim 13, wherein using the machine learning model comprises generating the proposed setup with one or more fixed teeth.

16. The method of claim 13, wherein using the machine learning model comprises generating the proposed setup with one or more pinned teeth.

17-18. (canceled)
